(19) United States
(12) Patent Application Publication
(10) Pub. No.: US 2018/0199889 A1
(43) Pub. Date: Jul. 19, 2018

(54) TRANSFER FUNCTION FOR TONOMETER SIGNALS

(71) Applicant: Microsoft Technology Licensing, LLC, Redmond, WA (US)

(72) Inventors: Jeremiah Wander, Redmond, WA (US); Sumit Basu, Redmond, WA (US); Daniel Morris, Bellevue, WA (US)

(73) Assignee: Microsoft Technology Licensing, LLC, Redmond, WA (US)

(21) Appl. No.: 15/406,553

(22) Filed: Jan. 13, 2017

(51) Int. Cl. A61B 5/00; A61B 5/021

(52) U.S. Cl. CPC A61B 5/7239; A61B 5/021; A61B 5/7275; A61B 5/7246; A61B 5/6831

(57) ABSTRACT

According to one embodiment of the present disclosure, a computing device is provided, comprising a processor configured to receive an input. The input includes a first pulse pressure signal obtained using a wearable tonometer affixed to a body of a user. The processor is further configured to apply a transfer function to the first pulse pressure signal, wherein the transfer function converts the first pulse pressure signal into a transformed pulse pressure signal. The transformed pulse pressure signal simulates a second pulse pressure signal of a handheld tonometer concurrently applied to the body of the user.

### BACKGROUND

Measurement of blood pressure has long been a clinical mainstay for assessing cardiovascular health. More recently, scientific evidence suggests that measurement of the entire arterial waveform, not just estimation of the peak and the trough of that waveform, can provide clinically meaningful information. Measurement of the entire arterial waveform can be achieved non-invasively via arterial tonometry, though this technique has historically been limited in use to in-clinic monitoring sessions.

Radial arterial tonometry can be performed in an ambulatory setting, but to address many of the challenges associated with continuous ambulatory wear, these tonometers are notably different than their handheld counterparts. Correspondingly, the nature of the signals recorded by ambulatory devices is different from the signals recorded by handheld devices, thus the data are not directly comparable.

### SUMMARY

According to one embodiment of the present disclosure, a computing device is provided, comprising a processor configured to receive an input. The input includes a first pulse pressure signal obtained using a wearable tonometer affixed to a body of a user. The processor is further configured to apply a transfer function to the first pulse pressure signal, wherein the transfer function converts the first pulse pressure signal into a transformed pulse pressure signal. The transformed pulse pressure signal simulates a second pulse pressure signal of a handheld tonometer concurrently applied to the body of the user.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 schematically shows a computing device, including a processor configured to apply a transfer function to a first pulse pressure signal, according to one embodiment of the present disclosure.

FIG. 2 shows an example depiction of a computing device being used with a wearable tonometer and a handheld tonometer, according to one embodiment of the present disclosure.

FIG. 3 shows a flowchart of a method for generating a transfer function for use with a computing device, according to one embodiment of the present disclosure.

FIG. 4 depicts a graph of an impulse response of an example transfer function, according to one embodiment of the present disclosure.

FIG. 5A depicts a graph of a magnitude response of an example transfer function, according to one embodiment of the present disclosure.

FIG. 5B depicts a graph of a phase response of an example transfer function, according to one embodiment of the present disclosure.

FIG. 6 depicts a graph of an input and output of an example transfer function, according to one embodiment of the present disclosure.

FIG. 7 shows a flowchart of a method for applying a transfer function to a first pulse pressure signal, according to one embodiment of the present disclosure.

FIG. 8 shows an example computing system, according to one embodiment of the present disclosure.

### DETAILED DESCRIPTION

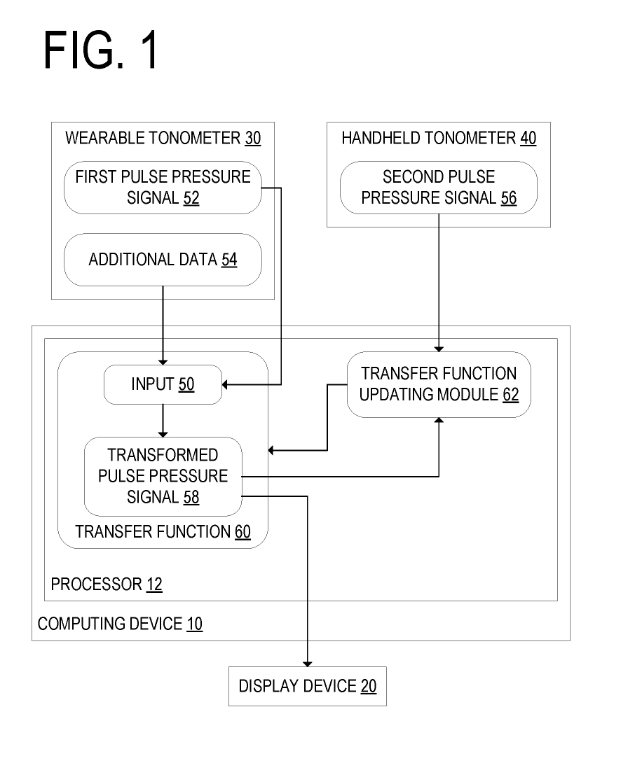

FIG. 1 schematically shows a computing device 10, including a processor 12. The processor 12 is configured to receive an input 50, wherein the input 50 includes a first pulse pressure signal 52 obtained using a wearable tonometer 30 affixed to a body of a user. The term “wearable tonometer” refers here to a device that is configured to measure a pulse pressure wave of pulse pressure in an artery of the user, and is affixed to the body of the user for ambulatory use. The wearable tonometer 30 may obtain the first pulse pressure signal 52 at a location selected from a group consisting of a radial artery, an ulnar artery, a femoral artery, a temporal artery, and an arcuate artery of the foot of a user. A wearable tonometer may be worn while the user performs everyday activities, as opposed to tonometers designed for use in a clinical setting only. The wearable tonometer 30 may be in the form of a band.

In addition to the first pulse pressure signal 52, the input 50 may include additional data 54 transmitted to the processor 12 from the wearable tonometer 30. For example, the input 50 may include timestamps. The processor 12 may be configured to receive the input 50 as a wired or wireless communication, and may receive the input 50 over a network.

The processor 12 is configured to apply a transfer function 60 to the first pulse pressure signal 52. The transfer function 60 converts the first pulse pressure signal 52 into a transformed pulse pressure signal 58. The transfer function 60 may also use the additional data 54 included in the input 50 along with the first pulse pressure signal 52 to generate the transfer function 60. The transformed pulse pressure signal 58 simulates a second pulse pressure signal 56 of a handheld tonometer 40 concurrently applied to the body of the user. The term “handheld tonometer” refers here to a device that is configured to measure a pulse pressure wave of pulse pressure in an artery of the user, but, unlike a wearable tonometer, is designed for handheld use rather than ambulatory use. The processor 12 may be configured to receive the second pulse pressure signal 56 as a wired or wireless communication, and may receive the second pulse pressure signal 56 over a network.

By simulating the second pulse pressure signal 56, the transformed pulse pressure signal 58 approximates the signal that would be received from the handheld tonometer 40, whether or not a second pulse pressure signal 56 measured using the handheld tonometer 40 is actually received by the processor 12 concurrently with the measurement of the first pulse pressure signal 52. The transformed pulse pressure signal 58 may be compared to a pulse pressure signal measured by the handheld tonometer 40 at a different time. The first pulse pressure signal 52 may thus be compared to pulse pressure measurements made earlier with the handheld tonometer 40, even when the first pulse pressure signal 52 is measured at a time when it would be impractical to make a pulse pressure measurement with the handheld tonometer 40.

When measurements with the wearable tonometer 30 and handheld tonometer 40 are made concurrently, the processor 12 may be configured to compare the transformed pulse pressure signal 58 to the second pulse pressure signal 56. This comparison allows the accuracy of the simulation made by the transfer function 60 to be evaluated. When the processor 12 compares the transformed pulse pressure signal 58 to the second pulse pressure signal 56, it may be configured to modify the transfer function 60 based on the comparison between the transformed pulse pressure signal 58 and the second pulse pressure signal 56. Modifying the transfer function 60 may be done using a transfer function updating module 62 that takes as inputs the transformed pulse pressure signal 58 and the second pulse pressure signal 56. By modifying the transfer function 60 based on such a comparison, the transfer function 60 may be calibrated subsequently to an initial generation of the transfer function 60. The transfer function 60 may be calibrated for use with a different wearable tonometer 30 and/or handheld tonometer 40 from those with which it was initially generated.

The transformed pulse pressure signal 58 may be conveyed for output on a display device 20. The processor 12 may be further configured to output other data, such as the first pulse pressure signal 52 and the second pulse pressure signal 56, for output on the display device 20.

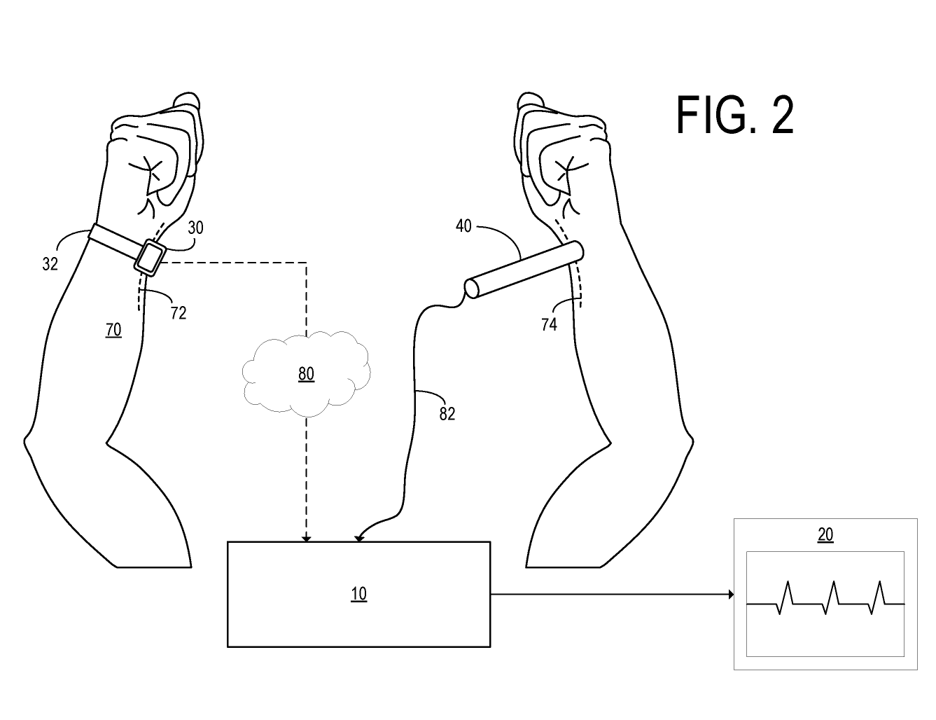

An example depiction of the computing device 10 being used with the wearable tonometer 30 and the handheld tonometer 40 is shown in FIG. 2. The computing device 10 is configured to receive measurements from the wearable tonometer 30 and the handheld tonometer 40. In the embodiment of FIG. 2, the first pulse pressure signal 52 is obtained from a different body location than the second pulse pressure signal 56. The wearable tonometer 30 measures a first pulse pressure wave in a first radial artery 72 of a user 70, and the handheld tonometer 40 measures a second pulse pressure wave in a second radial artery 74 of the user 70. The wearable tonometer 30 of FIG. 2 is affixed to skin proximate the first radial artery 72 of the user 70 by a band 32.

In the example embodiment of FIG. 2, pulse pressure signals are measured concurrently by the wearable tonometer 30 and the handheld tonometer 40. The wearable tonometer 30 transmits the first pulse pressure signal 52 to the computing device 10 wirelessly over a network 80. The handheld tonometer 40 transmits the second pulse pressure signal 56 to the computing device 10 via a wire 82. The computing device 10 transmits the transformed pulse pressure signal 58 for output on a display device 20.

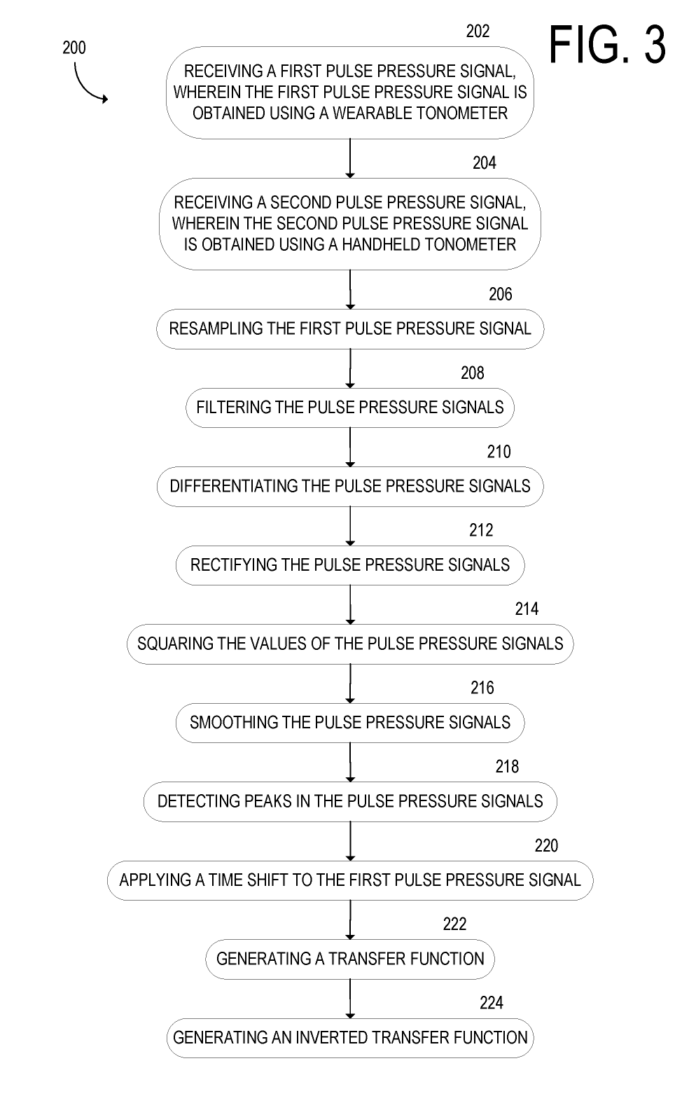

FIG. 3 shows a flowchart of a method 200 for generating a transfer function for use with a computing device. At step 202, the method includes receiving a first pulse pressure signal. The first pulse pressure signal is obtained using a wearable tonometer affixed to a body of a user, and is a measurement of a pulse pressure wave in an artery of the user. At step 204, the method includes receiving a second pulse pressure signal. The second pulse pressure signal is obtained using a handheld tonometer concurrently applied to the body of the user. The second pulse pressure signal may be measured at the same artery as the first pulse pressure measurement, or a different artery. The first pulse pressure signal and second pulse pressure signal are measured simultaneously for a period of time.

At step 206, the method for generating the transfer function may include resampling the first pulse pressure signal so that the sampling rate of the first pulse pressure signal is equal to a sampling rate of the second pulse pressure signal.

Next, the computing device may synchronize the signals received from the wearable tonometer and the handheld tonometer. Synchronizing the signals may be done by identifying systolic upstrokes and applying a time shift to the first pulse pressure signal so that the systolic upstrokes of the two signals align with each other. At step 208, the method may include filtering the first pulse pressure signal and the second pulse pressure signal to reduce high- and low-frequency noise. Filtering the pulse pressure signals may include performing Fourier transforms on them to separate high- and low-frequency components from other components of the signals or convolving the pressure signals with infinite impulse response or finite impulse response filters designed to remove high- and low-frequency components from the signals. At step 210, the method may include differentiating the first pulse pressure signal and the second pulse pressure signal. Differentiating the pulse pressure signals allows the computing device to determine when large upstrokes in the signals occur. At step 212, the method may include rectifying the first pulse pressure signal and the second pulse pressure signal to set values of the signals that are below some threshold value equal to that value. Rectifying the signals allows large upstrokes to be distinguished from smaller upstrokes that may not exceed the threshold value. At step 214, the method may include squaring the values of the first pulse pressure signal and the second pulse pressure signal. Squaring the values of the signals may make large upstrokes more easily distinguishable from smaller upstrokes. At step 216, the method may include smoothing the first pulse pressure signal and the second pulse pressure signal. At step 218, the method may include detecting a first plurality of peaks in the first pulse pressure signal and detecting a second plurality of peaks in the second pulse pressure signal.

Once the first and second plurality of peaks have been identified, the method may include, at step 220, applying a time shift to the first pulse pressure signal so that peaks of the first plurality of peaks occur at times that most closely match times at which peaks of the second plurality of peaks occur. Detecting the peaks in each pulse pressure signal may include determining when that signal exceeds a predetermined threshold. When a pulse pressure signal exceeds the predetermined threshold, the computing device may determine that a systolic upstroke has occurred. Applying the time shift may include generating a first sequence of times at which peaks occur in the first pulse pressure signal, and may further include detecting a second plurality of peaks in the second pulse pressure signal. The method may then include determining a value for the time shift such that, when the time shift is applied to the first sequence, the time shift maximizes a cross-correlation between the first pulse pressure signal and the second pulse pressure signal. The time shift may then be applied to the unmodified first pulse pressure signal in order to make it align with the unmodified second pulse pressure signal.

At step 222, the method includes generating a transfer function that, when applied to the first pulse pressure signal during the period of time for which the first and second pulse pressure signals were measured simultaneously, produces a transformed pulse pressure signal that simulates the second pulse pressure signal during the same period of time. The transfer function may be generated using regularized linear regression. Regularized linear regression sometimes may not be sufficient to produce an accurate simulation of the second pulse pressure signal from the first pulse pressure signal. Thus, in some embodiments, the transfer function may be generated using a nonlinear scaling function, which may be a single-parameter function.

The transfer function may be generated for a specific combination of a user, a wearable tonometer, and a handheld tonometer. Generating a transfer function for a specific combination of user, wearable tonometer, and handheld tonometer allows the transfer function to account for detector characteristics of the wearable tonometer and handheld tonometer, as well as anatomical and physiological characteristics of the user. Alternately, a generic transfer function that may be used with a wider range of users, wearable tonometers, and handheld tonometers may be generated. If a generic transfer function is generated, the first pulse pressure signal and second pulse pressure signal may be obtained from a mechanically simulated heartbeat.

At step 224, the method may include generating an inverted transfer function. When performed on the second pulse pressure signal during the period of time, the inverted transfer function may produce a second transformed pulse pressure signal that matches the first pulse pressure signal during the same period of time. Using the inverted transfer function, the first pulse pressure signal obtained from the wearable tonometer applied to the body of the user concurrently with the handheld tonometer may be simulated based on the second pulse pressure signal. This simulation may be performed even when a wearable tonometer is not applied to the body of the user. The inverted transfer function may allow a signal obtained from a handheld tonometer to be compared to a signal obtained from a wearable tonometer at an earlier time.

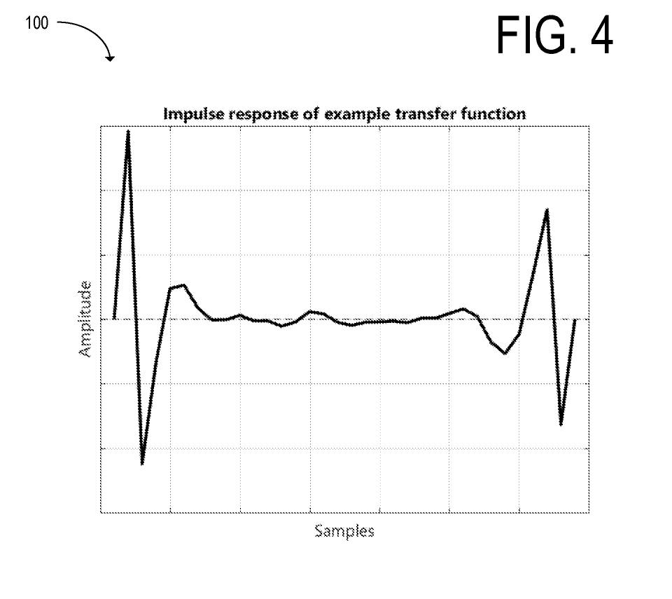

FIGS. 4-6 characterize an example transfer function. FIG. 4 depicts a graph 100 of the impulse response of an example transfer function. The example transfer function is configured to receive a plurality of samples of a pulse pressure signal and output a modified pulse pressure signal.

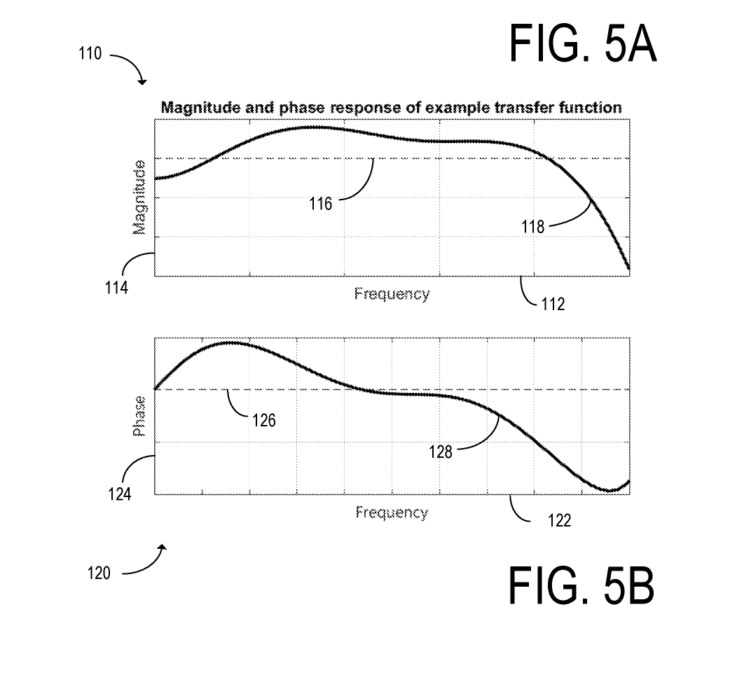

FIG. 5A depicts a graph 110 of a magnitude response of an example transfer function. The graph 110 shows the effect of the frequencies 112 of components of a pulse pressure signal on the magnitude 114 of the components of a transformed pulse pressure signal with the same frequencies 112. When the pulse pressure signal is obtained using a wearable tonometer, a pulse pressure wave may travel through one or more materials that attenuate components of the pulse pressure wave to an extent that depends on the frequencies 112 of those components. The extent to which a component is attenuated when measured using a wearable tonometer may be different from the extent to which that component is attenuated when measured using a handheld tonometer. The example transfer function accounts for this difference in attenuation. Due to the response properties of the example transfer function, for each frequency 112, the example transfer function modifies the magnitude 114 of the component with that frequency, with respect to a baseline magnitude 116. Magnitude 114 as a function of frequency 112 is plotted to form a curve 118.

FIG. 5B depicts a graph 120 of a phase response of an example transfer function. The graph 120 shows the effect of the frequencies 122 of components of a pulse pressure signal on the phase shifts 124 of the components of a transformed pulse pressure signal with the same frequencies 122. When the pulse pressure signal is measured by a wearable tonometer, the pulse pressure signal may pass through one or more materials which cause frequency-specific phase delays. As a result, components of the pulse pressure signal may be detected at different times from the times at which they would be detected using a handheld tonometer. For each frequency 122, the example transfer function applies a phase shift 124 to the component with that frequency, with respect to a baseline phase shift 126. Phase shift 124 as a function of frequency 122 is plotted to form a curve 128.

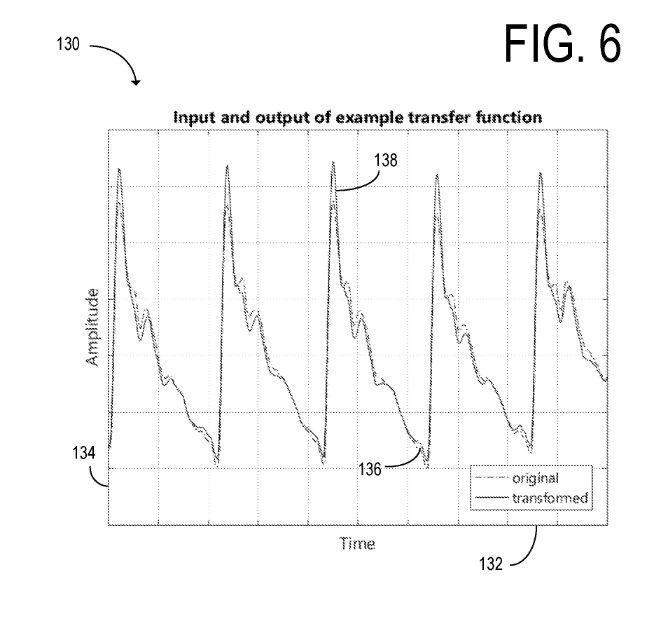

FIG. 6 depicts a graph 130 of an input and output of an example transfer function. The graph 130 shows the amplitude 134 of an original pulse pressure signal 136 and a transformed pulse pressure signal 138 as a function of time 132. When the example transfer function is applied to the original pulse pressure signal 136, it outputs the transformed pulse pressure signal. The example transfer function may apply changes in magnitude and phase to the original pulse pressure signal 136, as described above.

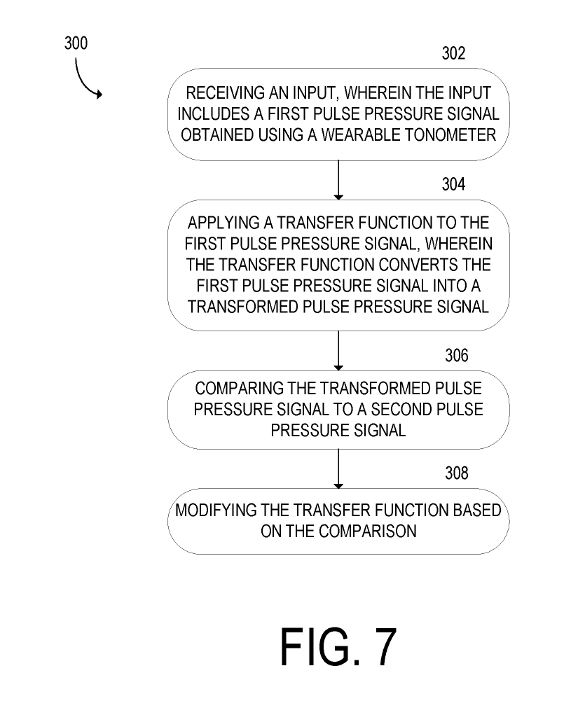

FIG. 7 shows a flowchart of a method 300 for use with a computing device. At step 302, the method includes receiving an input, wherein the input includes a first pulse pressure signal obtained using a wearable tonometer affixed to a body of a user. The wearable tonometer is configured to detect a pulse pressure wave of pulse pressure in an artery of the user. In addition to the first pulse pressure signal, the input may also include additional data obtained from the wearable tonometer.

At step 304, the method includes applying a transfer function to the first pulse pressure signal, wherein the transfer function converts the first pulse pressure signal into a transformed pulse pressure signal. The transformed pulse pressure signal simulates a second pulse pressure signal of a handheld tonometer concurrently applied to the body of the user. By applying the transfer function to the first pulse pressure signal, a simulation of a signal from a handheld tonometer may be generated even when a handheld tonometer is not concurrently applied to the body of the user. The transformed pulse pressure signal may be compared directly to signals obtained by the handheld tonometer at other times.

At step 306, when a second pulse pressure signal is measured concurrently with the first pulse pressure signal, the method may include comparing the transformed pulse pressure signal to the second pulse pressure signal. When the transformed pulse pressure signal is compared to the second pulse pressure signal, the method may include, at step 308, modifying the transfer function based on the comparison between the transformed pulse pressure signal and the second pulse pressure signal.

In some embodiments, the methods and processes described herein may be tied to a computing system of one or more computing devices. In particular, such methods and processes may be implemented as a computer-application program or service, an application-programming interface (API), a library, and/or other computer-program product.

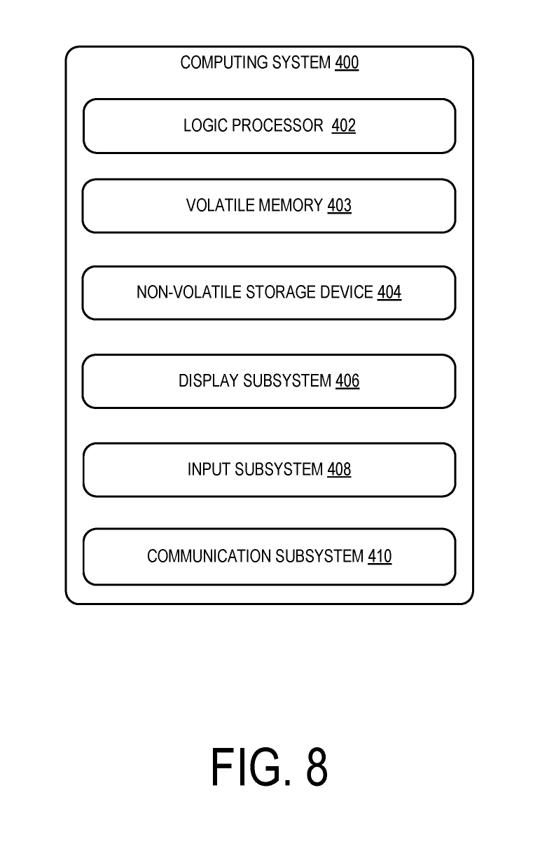

FIG. 8 schematically shows a non-limiting embodiment of a computing system 400 that can enact one or more of the methods and processes described above. Computing system 400 is shown in simplified form. Computing system 400 may embody the computing device 10 of FIG. 1. Computing system 400 may take the form of one or more personal computers, server computers, tablet computers, home-entertainment computers, network computing devices, gaming devices, mobile computing devices, mobile communication devices (e.g., smart phone), and/or other computing devices, and wearable computing devices such as smart wristwatches and head mounted augmented reality devices.

Computing system 400 includes a logic processor 402 volatile memory 403, and a non-volatile storage device 404. Computing system 400 may optionally include a display subsystem 406, input subsystem 408, communication subsystem 410, and/or other components not shown in FIG. 8.

Logic processor 402 includes one or more physical devices configured to execute instructions. For example, the logic processor 402 may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor 402 may include one or more physical processors (hardware) configured to execute software instructions. Additionally or alternatively, the logic processor 402 may include one or more hardware logic circuits or firmware devices configured to execute hardware-implemented logic or firmware instructions. Processors of the logic processor 402 may be single-core or multi-core, and the instructions executed thereon may be configured for sequential, parallel, and/or distributed processing. Individual components of the logic processor 402 optionally may be distributed among two or more separate devices, which may be remotely located and/or configured for coordinated processing. Aspects of the logic processor 402 may be virtualized and executed by remotely accessible, networked computing devices configured in a cloud-computing configuration. In such a case, these virtualized aspects are run on different physical logic processors of various different machines, it will be understood.

Non-volatile storage device 404 includes one or more physical devices configured to hold instructions executable by the logic processor 402 to implement the methods and processes described herein. When such methods and processes are implemented, the state of non-volatile storage device 404 may be transformed—e.g., to hold different data.

Non-volatile storage device 404 may include physical devices that are removable and/or built-in. Non-volatile storage device 404 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Non-volatile storage device 404 may include nonvolatile, dynamic, static, read/write, read-only, sequential-access, location-addressable, file-addressable, and/or content-addressable devices. It will be appreciated that non-volatile storage device 404 is configured to hold instructions even when power is cut to the non-volatile storage device 404.

Volatile memory 403 may include physical devices that include random access memory. Volatile memory 403 is typically utilized by logic processor 402 to temporarily store information during processing of software instructions. It will be appreciated that volatile memory 403 typically does not continue to store instructions when power is cut to the volatile memory 403.

Aspects of logic processor 402, volatile memory 403, and non-volatile storage device 404 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 400 typically implemented in software by a processor to perform a particular function using portions of volatile memory, which function involves transformative processing that specially configures the processor to perform the function. Thus, a module, program, or engine may be instantiated via logic processor 402 executing instructions held by non-volatile storage device 404, using portions of volatile memory 403. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 406 may be used to present a visual representation of data held by non-volatile storage device 404. The visual representation may take the form of a graphical user interface (GUI). As the herein described methods and processes change the data held by the non-volatile storage device, and thus transform the state of the non-volatile storage device, the state of display subsystem 406 may likewise be transformed to visually represent changes in the underlying data. Display subsystem 406 may include one or more display devices utilizing virtually any type of technology. Such display devices may be combined with logic processor 402, volatile memory 403, and/or non-volatile storage device 404 in a shared enclosure, or such display devices may be peripheral display devices.

When included, input subsystem 408 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor.

When included, communication subsystem 410 may be configured to communicatively couple various computing devices described herein with each other, and with other devices. Communication subsystem 410 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some embodiments, the communication subsystem may allow computing system 400 to send and/or receive messages to and/or from other devices via a network such as the Internet.

The following paragraphs provide additional support for the claims of the subject application. According to one aspect of the present disclosure, a computing device is provided, comprising a processor configured to receive an input. The input may include a first pulse pressure signal obtained using a wearable tonometer affixed to a body of a user. The processor may be further configured to apply a transfer function to the first pulse pressure signal. The transfer function may convert the first pulse pressure signal into a transformed pulse pressure signal. The transformed pulse pressure signal may simulate a second pulse pressure signal of a handheld tonometer concurrently applied to the body of the user.

According to this aspect, the transformed pulse pressure signal may be conveyed for output on a display device.

According to this aspect, the wearable tonometer may obtain the first pulse pressure signal at a location selected from a group consisting of a radial artery, an ulnar artery, a femoral artery, a temporal artery, and an arcuate artery of the foot of a user.

According to this aspect, the first pulse pressure signal may be obtained from a different body location than the second pulse pressure signal.

According to this aspect, the wearable tonometer may be in the form of a band.

According to this aspect, the processor may be configured to compare the transformed pulse pressure signal to the second pulse pressure signal.

According to this aspect, the processor may be configured to modify the transfer function based on the comparison between the transformed pulse pressure signal and the second pulse pressure signal.

According to another aspect of the present disclosure, a method for generating a transfer function for use with a computing device is provided. The method may comprise receiving a first pulse pressure signal. The first pulse pressure signal may be obtained using a wearable tonometer affixed to a body of a user. The method may further include receiving a second pulse pressure signal. The second pulse pressure signal may be obtained using a handheld tonometer concurrently applied to the body of the user. The first pulse pressure signal and second pulse pressure signal may be measured simultaneously for a period of time. The method may further include generating a transfer function that, when performed on the first pulse pressure signal during the period of time, produces a transformed pulse pressure signal that simulates the second pulse pressure signal during the same period of time.

According to this aspect, the method for generating the transfer function may include resampling the first pulse pressure signal so that the sampling rate of the first pulse pressure signal is equal to a sampling rate of the second pulse pressure signal.

According to this aspect, the method for generating the transfer function may include filtering the first pulse pressure signal and the second pulse pressure signal to reduce high- and low-frequency noise. The method may further include differentiating the first pulse pressure signal and the second pulse pressure signal. The method may further include rectifying the first pulse pressure signal and the second pulse pressure signal to set values of the signals that are below some threshold value equal to that value. The method may further include squaring the values of the first pulse pressure signal and the second pulse pressure signal. The method may further include smoothing the first pulse pressure signal and the second pulse pressure signal.

According to this aspect, the method for generating the transfer function may include detecting a first plurality of peaks in the first pulse pressure signal. The method may further include detecting a second plurality of peaks in the second pulse pressure signal. The method may further include applying a time shift to the first pulse pressure signal so that peaks of the first plurality of peaks occur at times that most closely match times at which peaks of the second plurality of peaks occur.

According to this aspect, detecting the peaks in each pulse pressure signal may include determining when that signal exceeds a predetermined threshold.

According to this aspect, applying the time shift may include generating a first sequence of times at which peaks occur in the first pulse pressure signal. Applying the time shift may further include generating a second sequence of times at which peaks occur in the second pulse pressure signal. Applying the time shift may further include determining a value for the time shift such that, when the time shift is applied to the first sequence, the time shift maximizes a cross-correlation between the first pulse pressure signal and the second pulse pressure signal.

According to this aspect, the transfer function may be generated using regularized linear regression.

According to this aspect, the transfer function may be generated using a nonlinear scaling function.

According to this aspect, the transfer function may be generated for a specific combination of a user, a wearable tonometer, and a handheld tonometer.

According to this aspect, the first pulse pressure signal and second pulse pressure signal may be obtained from a mechanically simulated heartbeat.

According to this aspect, the method may further include generating an inverted transfer function that, when performed on the second pulse pressure signal during the period of time, produces a second transformed pulse pressure signal that matches the first pulse pressure signal during the same period of time.

According to another aspect of the present disclosure, a method for use with a computing device is provided, comprising receiving an input. The input may include a first pulse pressure signal obtained using a wearable tonometer affixed to a body of a user. The method may further include applying a transfer function to the first pulse pressure signal. The transfer function may convert the first pulse pressure signal into a transformed pulse pressure signal. The transformed pulse pressure signal may simulate a second pulse pressure signal of a handheld tonometer concurrently applied to the body of the user.

According to this aspect, the method may include comparing the transformed pulse pressure signal to the second pulse pressure signal. The method may further include modifying the transfer function based on the comparison between the transformed pulse pressure signal and the second pulse pressure signal.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing device, comprising:
a processor configured to:
receive an input, wherein the input includes a first pulse pressure signal obtained using a wearable tonometer affixed to a body of a user; and
apply a transfer function to the first pulse pressure signal, wherein the transfer function converts the first pulse pressure signal into a transformed pulse pressure signal, wherein the transformed pulse pressure signal simulates a second pulse pressure signal of a handheld tonometer concurrently applied to the body of the user.

2. The computing device of claim 1, wherein the transformed pulse pressure signal is conveyed for output on a display device.

3. The computing device of claim 1, wherein the wearable tonometer obtains the first pulse pressure signal at a location selected from a group consisting of a radial artery, an ulnar artery, a femoral artery, a temporal artery, and an arcuate artery of the foot of a user.

4. The computing device of claim 1, wherein the first pulse pressure signal is obtained from a different body location than the second pulse pressure signal.

5. The computing device of claim 1, wherein the wearable tonometer is in the form of a band.

6. The computing device of claim 1, wherein the processor is configured to compare the transformed pulse pressure signal to the second pulse pressure signal.

7. The computing device of claim 6, wherein the processor is configured to modify the transfer function based on the comparison between the transformed pulse pressure signal and the second pulse pressure signal.

8. A method for generating a transfer function for use with a computing device, the method comprising:
receiving a first pulse pressure signal, wherein the first pulse pressure signal is obtained using a wearable tonometer affixed to a body of a user;
receiving a second pulse pressure signal, wherein:
the second pulse pressure signal is obtained using a handheld tonometer concurrently applied to the body of the user; and
the first pulse pressure signal and second pulse pressure signal are measured simultaneously for a period of time; and
generating a transfer function that, when performed on the first pulse pressure signal during the period of time, produces a transformed pulse pressure signal that simulates the second pulse pressure signal during the same period of time.

9. The method of claim 8, wherein the method for generating the transfer function includes:
resampling the first pulse pressure signal so that the sampling rate of the first pulse pressure signal is equal to a sampling rate of the second pulse pressure signal.

10. The method of claim 9, wherein the method for generating the transfer function includes:
filtering the first pulse pressure signal and the second pulse pressure signal to reduce high- and low-frequency noise;
differentiating the first pulse pressure signal and the second pulse pressure signal;
rectifying the first pulse pressure signal and the second pulse pressure signal to set values of the signals that are below some threshold value equal to that value;
squaring the values of the first pulse pressure signal and the second pulse pressure signal; and
smoothing the first pulse pressure signal and the second pulse pressure signal.

11. The method of claim 10, wherein the method for generating the transfer function includes:
detecting a first plurality of peaks in the first pulse pressure signal;
detecting a second plurality of peaks in the second pulse pressure signal; and
applying a time shift to the first pulse pressure signal so that peaks of the first plurality of peaks occur at times that most closely match times at which peaks of the second plurality of peaks occur.

12. The method of claim 11, wherein detecting the peaks in each pulse pressure signal includes determining when that signal exceeds a predetermined threshold.

13. The method of claim 11, wherein applying the time shift includes:
generating a first sequence of times at which peaks occur in the first pulse pressure signal;
generating a second sequence of times at which peaks occur in the second pulse pressure signal; and
determining a value for the time shift such that, when the time shift is applied to the first sequence, the time shift maximizes a cross-correlation between the first pulse pressure signal and the second pulse pressure signal.

14. The method of claim 8, wherein the transfer function is generated using regularized linear regression.

15. The method of claim 14, wherein the transfer function is generated using a nonlinear scaling function.

16. The method of claim 8, wherein the transfer function is generated for a specific combination of a user, a wearable tonometer, and a handheld tonometer.

17. The method of claim 8, wherein the first pulse pressure signal and second pulse pressure signal are obtained from a mechanically simulated heartbeat.

18. The method of claim 8, further comprising generating an inverted transfer function that, when performed on the second pulse pressure signal during the period of time, produces a second transformed pulse pressure signal that matches the first pulse pressure signal during the same period of time.

19. A method for use with a computing device, comprising:
receiving an input, wherein the input includes a first pulse pressure signal obtained using a wearable tonometer affixed to a body of a user; and
applying a transfer function to the first pulse pressure signal, wherein the transfer function converts the first pulse pressure signal into a transformed pulse pressure signal, wherein the transformed pulse pressure signal simulates a second pulse pressure signal of a handheld tonometer concurrently applied to the body of the user.

20. The method of claim 19, further comprising:
comparing the transformed pulse pressure signal to the second pulse pressure signal; and
modifying the transfer function based on the comparison between the transformed pulse pressure signal and the second pulse pressure signal.
